(19) United States
(12) Patent Application Publication
(10) Pub. No.: US 2026/0308568 A1
(43) Pub. Date: Oct. 8, 2026

(54) METHODS OF ENGINEERING CELLS WITH MUTATED GENOMES USING INTACT LARGE CARGO TRANSFER SYSTEMS

(71) Applicant: MET Biotechnology Inc., Culver City, CA (US)

(72) Inventors: TING-HSIANG WU, Culver City, CA (US); JASON HONG, Culver City, CA (US); TUNG-YU WU, Culver City, CA (US)

(21) Appl. No.: 19/483,024

(22) PCT Filed: May 9, 2024
(86) PCT No.: PCT/US2024/028631

§ 371 Date: Nov. 10, 2025

Related U.S. Application Data
(60) Provisional application No. 63/501,144, filed on May 9, 2023.

(51) Int. Cl. C12N 15/10; C12N 5/00; C12N 9/12; C12N 15/11; C12N 15/87; C12Q 1/6869

(52) U.S. Cl. CPC C12N 15/102; C12N 5/0006; C12N 9/1252; C12N 15/11; C12N 15/87; C12Q 1/6869; C12Y 207/07007

(57) ABSTRACT

Systems and methods method of cellular engineering are provided. The systems and methods can include transferring exogenous intact mitochondria into recipient cells. In some aspects, the systems and methods can include removing or depleting endogenous mitochondria from one or more recipient cells, mutating mtDNA within one or more donor cells, extracting mitochondria having mutated mtDNA from the one or more donor cells, transferring the mitochondria having mutated mtDNA into the one or more recipient cells, wherein the mitochondria are transferred intact, identifying one or more phenotypic changes of the one or more recipient cells, wherein the one or more phenotypic changes is associated with the transferred mitochondria, and isolating one or more of the recipient cells based on the identified phenotypic changes.

### PRIORITY CLAIM

This patent application claims priority to U.S. provisional patent application no. 63/501,144, titled “METHODS OF ENGINEERING CELLS WITH MUTATED GENOMES USING INTACT LARGE CARGO TRANSFER SYSTEMS,” and filed on May 9, 2023, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Endogenous production of compounds such as biological macromolecules is a facet of cellular activity required for basic function. The nuclear and mitochondrial genome supply instruction for the associated intracellular processes involved in these production mechanisms including maintenance of energy metabolism by mitochondria. Many of the cell products have therapeutic potential and research is focused on exploiting these natural production mechanisms. However, increasing the production demand on the cell is associated with substantially increased energy demands on the mitochondria.

Modulating production of these compounds may include artificial manipulation of the cell, genomic aberrations, xenobiotic stressors, natural and artificial instigation, etc. Considering natural mechanisms associated with these processes, genomic mutations may lead to loss of function, or hyperactivity either of which may lead to a disease state of the cell. Considering research and drug development, artificial manipulation may not provide the corresponding increase in energy production to support the increased demands on production mechanisms.

Cellular form and function are maintained based on the instruction provided by DNA/mtDNA. The natural function for a cell to produce these molecules requires a substantial amount of energy and when the demand increases the processes can be highly taxing on the cell. Accordingly, genomic modification of mtDNA is of great importance to optimize mitochondrial energy metabolism.

Current attempts to exploit mitochondrial activity include relatively ineffective and inefficient techniques of targeted genome editing or cellular culture optimization that are both overwhelmingly time consuming and expensive. Some of these examples include the use of mitochondrial targeted endonucleases such as mitoTALEN or mitoZFN. These methods are limited in their ability to delete specific targeted sequences of mtDNA. Similarly, CRISPR-Cas9 is very limited by the specific sequence and targets that may be deleted. Other methods may employ bacterial or viral vectors for targeted sequence editing such as bacterial cytidine deaminases DddA, generally capable of single base substitution. Each of these techniques are accompanied by limitations, negative side effects, and toxicity.

Although the mitochondrial genome is relatively small at approximately 16.5 kbp encoding 37, relative to the nuclear genes, identifying the specific targets necessary for the current editing techniques in combination with the robust mtDNA repair mechanisms, has prevented substantial progress in efficient methods of mitochondrial modification.

For these reasons, it would be desirable to provide improved systems and methods for editing and modifying mitochondria (e.g., mtDNA). It would be particularly desirable to provide efficient and effective modification to endogenous mitochondria for the optimization of cellular production of therapeutically beneficial compounds and, even more desirably, to provide methods that offer broad opportunity to identify and effect one or more advantageous modifications in mitochondrial activity. At least some of these objectives will be met by the various embodiments that follow.

### SUMMARY OF THE DISCLOSURE

Described herein are methods for engineering cellular activity and function including modulating cellular activity and function by transferring intact organelles or cargo from one or more donor cells into one or more recipient cells.

In general, a method of cellular engineering using exogenous intact mitochondria may comprise the steps of removing endogenous mitochondria from one or more recipient cells. DNA (e.g., mtDNA) is mutated within one or more donor cells. Then, the method continues with extracting mitochondria having mutated mtDNA from the one or more donor cells, transferring the extracted mitochondria into the one or more recipient cells, identifying one or more phenotypic changes of the one or more recipient cells, and isolating one or more of the recipient cells based on the identified phenotypic changes.

This and other examples may include any of the following. The mitochondria can be transferred intact. Intact transfer of mitochondria may be achieved using one or more large-cargo transfer systems of techniques. For example, the mitochondria having one or more mutations in the mtDNA are extracted, isolated, and transferred intact. wherein the one or more phenotypic changes is associated with the transferred mitochondria. The step of mutating mtDNA within the one or more donor cells may include contacting the one or more donor cells with one or more agents configured to modulate one or more mitochondrial polymerases. For example, the one or more donor cells may be poly mutated cells. The method may further include the step of proliferating the one or more donor cells before extracting mitochondria therefrom. Proliferating may include cloning, growing, or otherwise increasing the population of the one or more donor cells. The method may further include the step of proliferating the one or more recipient cells after transferring the extracted mitochondria. Proliferating may include cloning, growing, or otherwise increasing the population of the one or more donor cells.

In some examples, the one or more phenotypic changes may be caused by the transferred mitochondria. the one or more donor cells comprise at least one mtDNA mutation caused by polymerase gamma (e.g., mutated polymerase gamma). In some examples, transferring the extracted mitochondria intact into the one or more recipient cells comprises generating one or more pores within a cellular membrane of the one or more recipient cells, pressurizing a flow of the extracted mitochondria into the one or more recipient cells through the one or more pores. In some examples, transferring the extracted mitochondria intact into the one or more recipient cells comprises disrupting a cellular membrane of the one or more recipient cells with a photothermal-based system, wherein the disrupted cellular membrane is configured to permit the transfer of the extracted mitochondria into the one or more recipient cells. The step of generating one or more pores within the cellular membrane of the one or more recipient cells may comprise growing one or more recipient cells on a porous silicon chip, contacting the silicon chip with a laser configured to increase a temperature of the silicon chip. In some examples, the temperature can be configured to disrupt a cellular membrane of the one or more recipient cells grown thereon.

In some examples, the method may further include the steps of cloning the one or more isolated recipient cells and sequencing mtDNA from the isolated one or more recipient cells. The sequenced mtDNA can include one or more different nucleotide sequences than the removed endogenous mitochondria.

This and other examples may include any of the following. The step of removing the endogenous mitochondria may comprise depleting the one or more recipient cells of endogenous mitochondria. Protein production is increased in the one or more recipient cells based on the transferred mitochondria. Growth rate of the one or more recipient cells is increased based on the transferred mitochondria. Exosome production is increased in the one or more recipient cells based on the transferred mitochondria. ATP synthesis is increased in the one or more recipient cells based on the transferred mitochondria. One or more metabolic processes is increased in the one or more recipient cells based on the transferred mitochondria.

This and other examples may include any of the following. Protein production is decreased in the one or more recipient cells based on the transferred mitochondria. Growth rate of the one or more recipient cells is decreased based on the transferred mitochondria. Exosome production is decreased in the one or more recipient cells based on the transferred mitochondria. ATP synthesis is decreased in the one or more recipient cells based on the transferred mitochondria. One or more metabolic processes is decreased in the one or more recipient cells based on the transferred mitochondria.

In general, a method of engineering mtDNA may include sequencing DNA of one or more cells, modifying mitochondria in the one or more cells as described herein, identifying one or more phenotypic changes in the one or more cells, sequencing the DNA of the one or more identified cells, comparing the sequenced DNA of the one or more cells against the sequenced DNA of the one or more identified cells, generating a synthetic mtDNA sequence according to one or more changes in the DNA of the one or more identified cells, wherein the synthetic mtDNA is associated with the one or more identified phenotypic changes.

In general, a method of modulating mitochondrial activity in a cell may include depleting native mitochondria from one or more recipient cells, mutating mtDNA within one or more donor cells, extracting mitochondria from the one or more donor cell, wherein the extracted mitochondria are intact and isolated, transferring the extracted mitochondria into the one or more recipient cells, wherein the mitochondria are transferred intact, identifying one or more phenotypic changes of the one or more recipient cells, wherein the one or more phenotypic changes is associated with the transferred mitochondria, and isolating one or more of the recipient cells based on the identified phenotypic changes.

This and other examples may include any of the following. The step of mutating mtDNA within the one or more donor cells can comprise contacting the one or more donor cells with a chemical agent configured to mutate mtDNA. The method may further include growing the one or more donor cells having the mutated mtDNA before extracting mitochondria therefrom. The method may further include growing the one or more recipient cells after transferring the extracted mitochondria. The transferred mitochondria can be configured to cause the one or more phenotypic changes. The one or more donor cells may comprise at least one mtDNA mutation in polymerase gamma. The method may also include cloning the one or more isolated recipient cells and sequencing mtDNA from the isolated one or more recipient cells, wherein the sequenced mtDNA comprises one or more different nucleotide sequences than the removed endogenous mitochondria.

In some examples, transferring the extracted mitochondria intact into the one or more recipient cells may comprise generating one or more pores within a cellular membrane of the one or more recipient cells, pressurizing a flow of the extracted mitochondria into the one or more recipient cells through the one or more pores. In some examples, transferring the extracted mitochondria intact into the one or more recipient cells can comprise disrupting a cellular membrane of the one or more recipient cells with a photothermal-based system, wherein the disrupted cellular membrane is configured to permit the transfer of the extracted mitochondria into the one or more recipient cells. Generating one or more pores within the cellular membrane of the one or more recipient cells may comprise growing one or more recipient cells on a porous silicon chip, contacting the silicon chip with a laser configured to increase a temperature of the silicon chip, wherein the temperature is configured to disrupt a cellular membrane of the one or more recipient cells grown thereon.

In general, a method of modulating protein production by a cell may include depleting native mitochondria from one or more recipient cells and mutating mtDNA within one or more donor cells. Then, extracting mitochondria from the one or more donor cell, wherein the extracted mitochondria are intact and isolated, transferring the extracted mitochondria into the one or more recipient cells, wherein the mitochondria are transferred intact, identifying an increase in production of one or more proteins from the one or more recipient cells, wherein the increase in production of one or more proteins is associated with the transferred mitochondria and isolating one or more of the recipient cells based on the increased protein production.

In general, a method of modulating cellular production of one or more proteins may include depleting a mitochondria population from one or more recipient cells, transferring intact mitochondria from one or more mutated donor cells to the one or more recipient cells, wherein the intact mitochondria comprise one or more mutations in mtDNA compared to a wild-type mtDNA of non-mutated donor cells.

In general, a method of mitochondrial engineer using large-cargo transfer systems may include depleting endogenous mitochondria from one or more recipient cells, mutating mtDNA within one or more donor cells, extracting isolated mitochondria having mutated mtDNA from the one or more donor cells, transferring the extracted mitochondria into the one or more recipient cells using a large-cargo transfer system, wherein the extracted mitochondria are transferred into the one or more recipient cells intact, identifying one or more phenotypic changes of the one or more recipient cells, wherein the one or more phenotypic changes is associated with the transferred mitochondria, and isolating one or more of the recipient cells based on the identified phenotypic changes.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is an illustration showing examples of cellular activity that may be modulated by the systems and methods described herein.

FIG. 2 shows an exemplary illustration of mitochondrial transfer according to the systems and methods described herein.

FIG. 3 shows an exemplary illustration of mitochondrial transfer including engineering and modification to induce mutagenesis in mitochondria derived from donor cell lines for transfer, according to the systems and methods described herein.

FIG. 4 shows an example of changes in antibody production yields of engineered cell lines based on the transfer of intact mitochondria according to the systems and methods described herein.

FIG. 5 shows and example changes in exosome production yields of engineered cell lines based on the transfer of intact mitochondria according to the systems and methods described herein.

FIG. 6 shows a flow diagram of an exemplary method related to engineering cells according to one or more methods described herein.

FIG. 7 shows a flow diagram of an exemplary method related to engineering cells according to one or more methods described herein.

### DETAILED DESCRIPTION

Genomic engineering of a cell may include the transfer of intact or substantially intact cellular components using large cargo delivery systems. Considering the complex nature of a eukaryotic genome and even more complex the unique mitochondrial genome, engineering, generating, identifying or otherwise modifying cellular activity is substantially expedited by the methods and systems described herein.

Any of the methods or techniques described herein may be performed with a cellular transfer platform. The cells described herein may be cell cultures or cell samples. In some aspects, the cell cultures or cell samples may be suspended in a medium, such as a cell culture media or growth medium, which may include a liquid or gel that supports the growth of microorganisms.

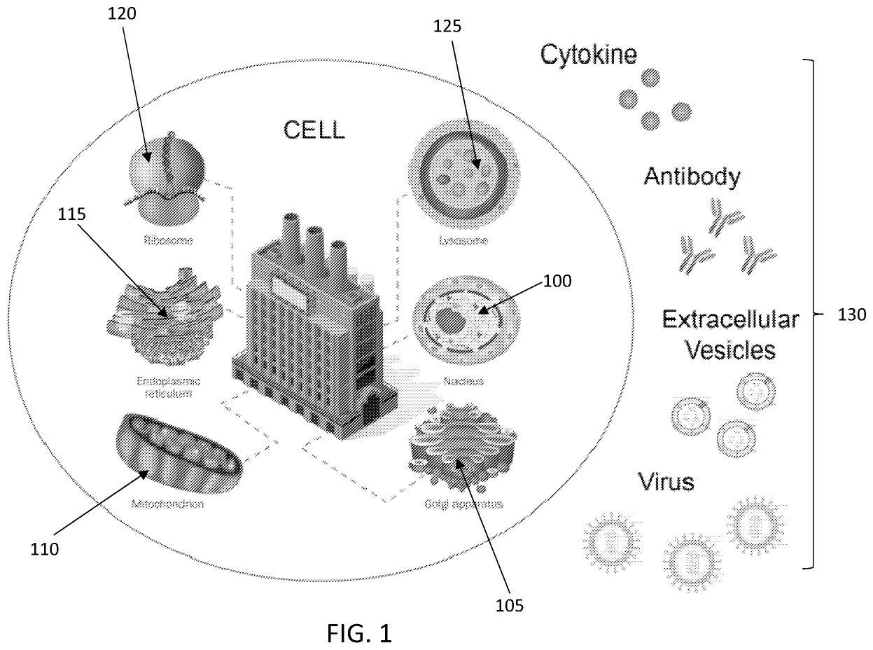

For example, phenotypic characteristics of a cell may include intracellular, intercellular and extracellular activities from molecule production and cell lifecycle to metabolic processes and intercellular signaling. As illustrated in FIG. 1, a cell has complex infrastructure comprising organelles such as the nucleus 100, golgi apparatus 105, mitochondria 110, endoplasmic reticulum 115, ribosomes 120, lysosomes 125, etc. One or more of these internal components can be involved in complex processes resulting in the production of compounds 130 including but not limited to cytokines, antibodies, extracellular vesicles, viruses, etc.

Engineering and modulating cell phenotypic characteristics, as described herein, can include the transfer (e.g., diffusion) of modified exogenous elements into a recipient cell for analysis, isolation, growth and exploitation of resulting changes to the native functions of the recipient cell. Accordingly, methods described herein may include depleting (e.g., removing) mitochondria from one or more first cells (e.g., a recipient cell). Exogenous mitochondria can be extracted, isolated, generated, obtained and/or derived from one or more second cells (e.g., donor cells). The exogenous mitochondria removed from the second or donor cells can then be transferred into the recipient cells that are then cloned, cultivated, grown, or otherwise allowed to proliferate including the exogenous mitochondria therein. The recipient cells may be analyzed, identified, selected, categorized, or otherwise evaluated based on changes in the activity (e.g., function) of the cells. Identified (e.g., identifiable) changes in recipient cell function, morphology, and/or any phenotypic characteristic may provide motivation for categoric separation and further comparative analysis of mitochondrial DNA (mtDNA) associated with and/or attributable to the phenotypic changes.

In some aspects, the cells may be analyzed with a microscope based system, or any other system for evaluating, analyzing, or categorizing cells or cell cultures.

The recipient cells can be configured in a mitochondrial-null state. For example, removing (e.g., depleting) mitochondria from recipient cells may include a method of depleting mtDNA from recipient cell with one or more chemical agents, genetic modifications, physical methods, or a combination thereof. For example, depleting mtDNA from recipient cells may include induced mitophagy within recipient cells sufficient to deplete all or less than all of the endogenous mitochondria.

In some examples, removing the mitochondria from the recipient cells may include removing all or less than all of the endogenous (e.g., native) mitochondria from the cell. In some examples, the amount of mitochondria removed from the recipient cells may be a percentage of the total mitochondria within the cell. For example, removing the mitochondria may include removing 100%, 90%, 80%, 70%, 60%, 50%, 40%, 30%, 20%, 10%, or less, or any percentage therebetween. In some examples, removing the mitochondria may include modifying the recipient cells to be mitochondria-null recipient cells.

In some examples, recipient cells may be initially engineered without mitochondria. For example, the one or more first cells (e.g., recipient cells) may be cultured or otherwise prepared and maintained in a mitochondria-null state. In some examples, a method for engineering mtDNA and cellular activities may include the step of generating one or more recipient cells to have a low level of mitochondria. In some examples, a method for engineering mtDNA and cellular activities may include the step of generating one or more recipient cells to have a non-detectable level of mitochondria. In some examples, a method for engineering mtDNA and cellular activities may include the step of generating one or more recipient cells to have a no level of mitochondria.

The exogenous mitochondria used in engineering recipient cells may be extracted from one or more donor cells. In some examples, mitochondrial extraction and isolation may be achieved through any means configured to isolate and extract intact mitochondria (e.g., differential centrifugation). mtDNA may be analyzed (e.g., sequenced) after extraction, before extraction or both. Donor cells may be a different type of cell or cell line with homologous DNA (e.g., mtDNA) to recipient cells. In some examples, donor cell genomic homology may be 1%, 5%, 10%, 15%, 20%, 25%, 30%, 35%, 40%, 45%, 50%, 55%, 60%, 65%, 70%, 75%, 80%, 85%, 90%, 95%, 100% or any percentage therebetween homologous to the recipient cell DNA. In some examples, exogenous mitochondria may be extracted from donor cells as wild-type (e.g., native) mitochondria. In some examples, donor cells may be modified (e.g., engineered) to provide one or more mutations in mtDNA encoding the mitochondria of the donor cells. In some examples, engineered donor cells may have genomic homology being 1%, 5%, 10%, 15%, 20%, 25%, 30%, 35%, 40%, 45%, 50%, 55%, 60%, 65%, 70%, 75%, 80%, 85%, 90%, 95%, 100% or any percentage therebetween homologous to the wild-type donor cell mtDNA.

Mutations in the donor cell genome (e.g., mtDNA) may be natural or artificial mutations that may impact form, function, and/or activity of one of more proteins. In some examples, the donor cell lines may be maintained, cultivated, grown, selected or otherwise used in any of the methods described herein based on the nature, frequency, concentration, quantity or other characteristics related to the mutations within the donor cell genome. In some examples, the donor cells may be subjected to or contacted by one or more agents configured to induce genomic mutations. In some examples, mutations may be induced by one or more chemical agents configured to mutate genomic material of one or more of the donor cells.

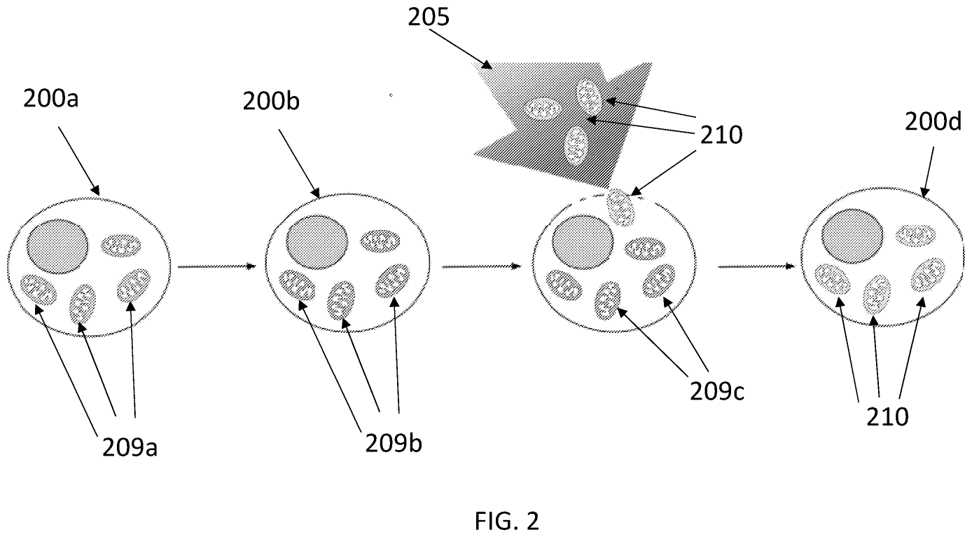

FIG. 2 illustrates a graphical representation of mitochondrial transfer from one or more donor cells (not shown) to one or more recipient cells 200a-200d. As illustrated, recipient cell 200a comprises native endogenous mitochondria 209a prior to undergoing one or more processes configured to deplete the mitochondria therein. Recipient cell 200b is shown with depleted endogenous mtDNA after this depletion process. With the mitochondria removed or depleted, recipient cell 200b is in a mitochondria-null state. For example, mitochondria 209b have been depleted or otherwise removed from recipient cell 200b.

Still referring to FIG. 2, a mitochondria transfer platform 205 as further described herein can be used to transfer exogenous mitochondria 210 from one or more donor cells (not shown) to the recipient cells 200a-200d. As the mitochondria cells are transferred into the recipient cells, the cells may include a combination of exogenous mitochondria 210 and depleted mitochondria (e.g., 209b/209c). As described herein, the exogenous mitochondria 210 may have different mtDNA compared to the endogenous mitochondria 209a. Here, the mtDNA of the exogenous mitochondria 210 from the donor cells may have a singular, or known number of mutations. FIG. 2 shows a fully engineered cell 200d with exogenous mitochondria 210 and mtDNA.

In some examples, one or more chemical agents may be configured to provide targeted, expected, anticipated, depletion, etc. mutations in the exogenous mitochondria before, during, or after the transfer process. In some aspects, depletion, mutation, and/or transfer of donor and/or recipient cells can be performed with a cellular engineering platform or system, which can include cell cultures, platforms, containers, or any other features required for holding cells, mutating cells, extracting cells, transferring cells, depleting cells, and/or evaluating cells. For example, the cellular engineering platform or system can include one or recipient cell cultures or containers configured to receive a first chemical agent for removing or depleting endogenous mitochondria from one or more recipient cells. The platform or system can further include one or more donor cell cultures or containers configured to receive a second chemical agent for mutating mtDNA within one or more donor cells. In some aspects, the system or platform can include an extraction apparatus configured for extracting mitochondria having mutated mtDNA from the one or more donor cells. In additional aspects, the platform or system can include a transfer platform or system (e.g., a BLAST system) configured to transfer the mitochondria having mutated mtDNA into the one or more recipient cells. Additionally, the platform or system can include an evaluation device (e.g., a microscope) configured for identifying one or more phenotypic changes of the one or more recipient cells, wherein the one or more phenotypic changes is associated with the transferred mitochondria. In other aspects, the system can include one or more isolation cultures or containers for isolating one or more of the recipient cells based on the identified phenotypic changes.

In some examples, the cellular engineering platform may employ chemicals to deplete or induce mutations in the cells. For example, Ethidium bromide may be used to deplete or induce mutations in mtDNA of known or expected significance. In some examples mutations may be induced to one or more gene encoding proteins involved in DNA damage repair or other complex metabolic processes that can be associated with the induction or precipitation of unpredictable and random mutations (e.g., mtDNA mutations). Any of the methods described herein may be configured to evaluate the impact of a plurality of induced mutations to genomic material of the donor cell (e.g., donor cell mtDNA mutations). In some examples, mutations in the mitochondria being transferred into the recipient cells may be unknown. For example, the nature of the mutations (e.g., single nucleotide polymorphisms, insertions, deletions, substitutions, etc. or a combination thereof) may be unknown. In some examples, any method described herein may be configured to process one or more unknown mutations associated with the large cargo being transferred into the recipient cells.

In some examples, donor cells may have one or more mutations in a gene or genes related to DNA repair mechanisms such as DNA polymerases. For example, POLG is a gene encoding a catalytic subunit of the mtDNA polymerase, polymerase gamma. Polymerase gamma is involved in base excision repair (BER) mechanisms. Impaired polymerase gamma function can be associated with downstream mutations to mtDNA based on inefficiency or inactivity with BER mechanisms. Accordingly, donor cells may have a plurality of mutations in the mtDNA. Mitochondria from these cells having random mutations in the mtDNA can provide a foundation for large-scale analysis of mtDNA changes and their impact on phenotypic characteristics.

In any of the embodiments described herein, the transfer platform can be configured for the efficient delivery of cellular cargo such as mitochondria into donor cells. The transfer platform can include physical approaches that typically involve disruption of the cellular membrane to create transient pores in the recipient cells followed by cargo delivery across the transient pores before they reseal. Many techniques are known for formation of transient pores, including electroporation, sonoporation, optoporation, optofluidics, and microfluidic channels. Any of these techniques may be used to transfer mitochondria from donor cells into the recipient cell via these pores in the cell membrane. In some examples, the mitochondria are transferred intact. For example, intact mitochondria can be extracted from the donor cell and transferred into the recipient cell. Recipient cells can be cultured after receiving the donor cell mitochondria.

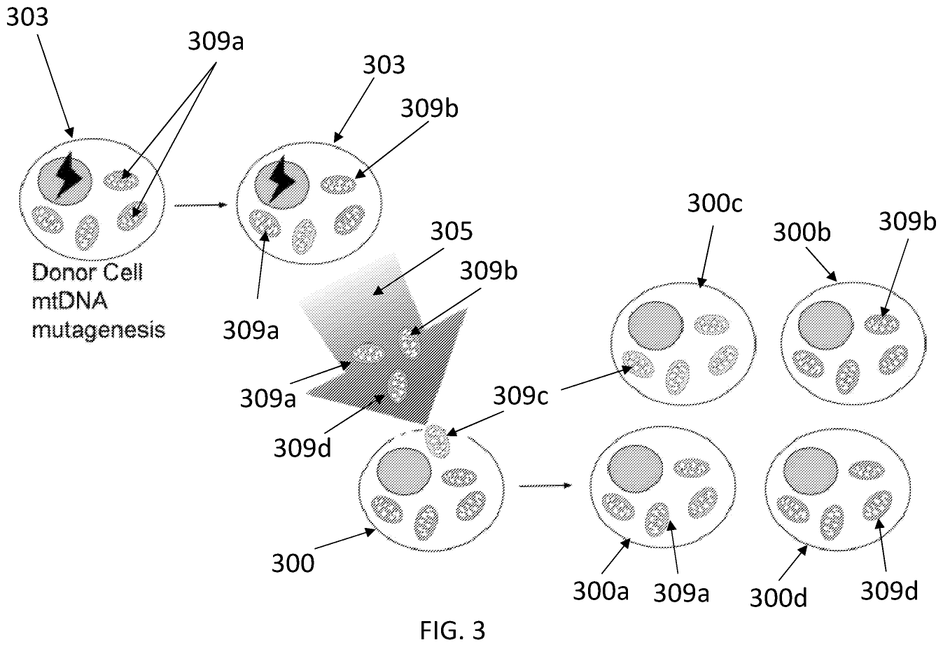

FIG. 3 illustrates a graphical representation of mitochondrial transfer from one or more donor cells 303 to one or more recipient cells 300, 300a, 300b, 300c, and 300d, according to methods described herein. In FIG. 3, donor cell 303 is subjected to one or more techniques, methods, processes, etc. configured to produce mtDNA mutagenesis in the cell, including one or more mutations, aberrations, or changes in the genomic signature or information relating to one or more donor cell components. For example, native endogenous mitochondria 309a are mutated as a result of the mutagenesis technique, method, process, etc. applied to the donor cell 303, resulting in the donor cell having one or more mutated mitochondria 309b, 309c, and 309d, with optionally unmutated or native mitochondria 309a still remaining in the cell.

Next, a recipient cell 300 having been depleted of endogenous mitochondria (e.g., such as recipient cell 200b in FIG. 2) is then prepared for large cargo transfer via transfer platform 305 according to any of the methods described herein.

In some examples, the recipient cells, or mitochondria from the donor cells, may be isolated, separated, and purified for evaluation of phenotypic changes based on the donor cell mitochondria transferred therein. In some examples, extraction, isolation, and purification techniques may be used to prepare the mitochondria derived from the donor cells. For example, isolation of the intact mitochondria of intact may include lysing the donor cells to release intracellular components including the intact mitochondria. In some examples, one or more tolls may be used to mechanically lyse the donor cells (e.g., a dounce homogenizer or tissue grinder). In some examples, the donor cells may be chemically lysed to release intracellular components including the intact mitochondria. For example, a detergent may be used to open the plasma membrane of a cell followed by applying force (e.g., mechanical force) to facilitate disrupting the cells and release all the intracellular cellular contents.

For example, after mutations in the mtDNA of the donor cells are established, intact mitochondria can be extracted from the donor cells and isolated in preparation for transfer to the recipient cells. In some examples, the mitochondria are intact or substantially intact. Multiple engineered cell lines with different mtDNA can be transferred to the donor cells. For example, in FIG. 3, the transfer platform 305 can facilitate the transfer of mitochondria 309a to cell 300a, mitochondria 309b to cell 300b, mitochondria 309c to cell 300c, and mitochondria 309d to cell 300d. Multiple lines of mtDNA engineered cells can be obtained in one or few transfers. This allows for screening of multiple mtDNA cell lines for a desired cellular phenotype. In some aspects, this can be combined with artificial intelligence or machine learning algorithms to identify mtDNA sequences or other phenotype correlation with the target function.

Any of the methods described herein may include the use of a large-cargo delivery system. In some examples, the mitochondria are cargo selected for transfer after they have been isolated from one or more donor cells. Some examples of large-cargo delivery systems may include one or more mechanisms configured to disrupt cell membranes (e.g., plasma membranes) to provide pores or openings in the membrane sufficient to facilitate the transfer (e.g., diffusion, passage, insertion, etc.) of mitochondria therethrough. The pores in the plasma membrane of the recipient cells may be provided by electrical techniques, acoustic techniques, thermal techniques, chemical techniques optical techniques, etc. or a combination thereof.

Some examples of large-cargo transfer systems and methods may be related to Biophotonic Laser Assisted Surgery Tool (BLAST), a transfer platform that includes culturing recipient cells on a silicon chip that can be thermally modified with a pulsed laser to create microcavitation bubbles that form pores on the surface of the cells allowing for the donor particles to enter the cell via pressurized flow without causing permanent damage. For example, mitochondria on a BLAST platform may include culturing recipient cells on a silicon chip. The chip may be loaded with the mitochondria as the large cargo to be delivered into the recipient cells. A laser (e.g., a pulse laser) can be triggered to scan the chip and generate pores in the recipient cells cultured thereon based on differences in heat across the surface of the chip in contact with the recipient cell membrane. Pressure is applied and configured to transfer (e.g., diffuse) the mitochondria through the pores directly into the interior of the cell (e.g., the cytoplasm). Additional details on the BLAST platform can be found in U.S. Pat. No. 10,472,651, which is incorporated herein by reference in its entirety.

Additional techniques include the MitoPunch platform which is based on the BLAST platform and includes a mechanical plunger to physically deform a pliable polydimethylsiloxane (PDMS) reservoir containing isolated mitochondria suspended in phosphate-buffered saline. Plunger activation propels the suspended cargo to force mitochondria into the cytosol of recipient cells.

In some examples, pressure (e.g., pressurized fluid) may be applied to transfer cargo into recipient cells. The amount of pressure applied may be in a range from 0.01 PSI or greater. For example, the amount of pressure applied may be 1 psi, 2 psi, 3 psi, 4 psi, 5 psi, or more of any amount of pressure therebetween.

In some examples, the pores generated by the methods described herein may be 0.01 micrometer (μm) or greater. In some examples, the pores generated by the methods described herein may be 0.01 μm or less. For example, a pore generated by a method described herein may be between 1 μm and 3 μm In some examples, pore size may be optimized based on the cargo being delivered (e.g., mitochondria) and may be 0.5 μm, 1 μm, 2 μm, 3 μm, 4 μm, 5 μm, or greater or any size therebetween. In some examples, pore density may be the quantity of pores generated per cell. In some examples, the pore density may be the quantity of pores generated per area of the cell. In some examples, the pore density may be 1 or more per cell. For example, pore density generated by the methods described herein may be 1 pore and 10 pores per cell. In some examples, the pore density may be 10 pores per cell or more.

In any method described herein, the transfer of cargo (e.g., mitochondria) into the recipient cell may result in the intact cargo being undamaged during the transfer process. For example, mitochondria being transferred into the recipient cell may maintain their form without sustaining any damage. In some examples, the cargo being transferred into the recipient cell may be damaged (e.g., impacted by the transfer process), but may not present any change in function or activity. For example, mitochondrial transfer accordingly to any method described herein may provide for the exogenous mitochondria being transferred into the recipient cell and incorporated into the recipient cell function or activity. In some examples, the recipient cells may rely on the transferred mitochondria for processes generally associated with the native mitochondria. For example, the transferred exogenous mitochondria may participate in the energy metabolism and production of adenosine triphosphate (ATP) for the recipient cell.

Engineering the recipient cells with the transferred mitochondria derived from the donor cells may then be cultured and grown. The engineered recipient cells may exhibit phenotypic changes related to their function or activities based on the change or impact of the exogenous mitochondria transferred therein. For example, the mitochondria transferred into the recipient cells may be configured to increase energy metabolism and ATP synthesis providing increased energy for recipient cellular activities. The engineered recipient cells can be analyzed and one or more phenotypic changes, genotypic changes, or a combination thereof (e.g., cell product yield, growth/proliferation rates, changes to gene expression, cell function, other phenotypic characteristics, etc.) may be identified. For example, recipient cell production of one or more proteins may be increased, decreased, or a combination thereof.

In some examples, engineered recipient cells may be selected and isolated based on common changes to the cellular activity (e.g., as caused by the exogenous mitochondria).

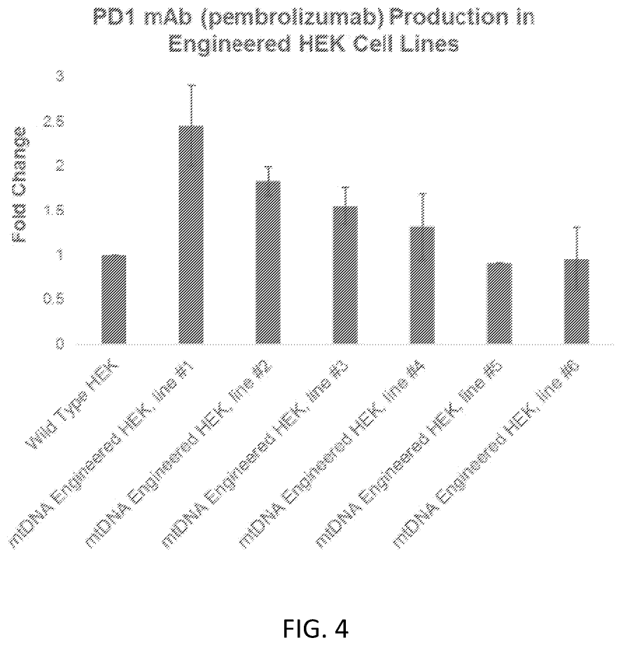

In FIG. 4, examples of the impact on antibody production in human embryonic kidney (HEK) cells is shown as compared to several engineered cell lines having exogenous intact mitochondria transferred therein according to one or more methods described herein. In particular, six engineered cell lines have been isolated after receiving the exogenous mtDNA from one or more donor cells. Engineered HEK cell line #1 indicates the most substantial increase in the production of PD1 compared to all other engineered lines including the wild-type (WT) HEK cells. As described herein, specific mutations in the mtDNA from the donor cells may be random and the impact observable during analysis of the engineered cells having received different mutations in the mtDNA associated with the intact mitochondria transferred therein.

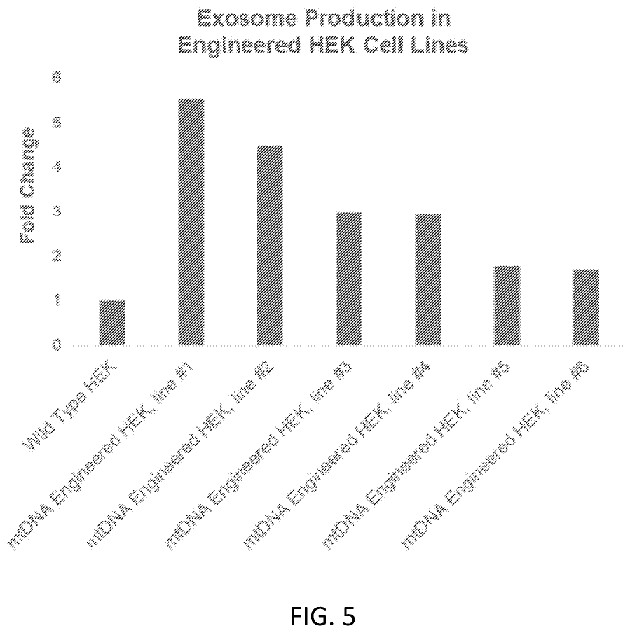

Similarly, FIG. 5 illustrates results regarding the changes in exosome production in engineered HEK cells according to one or more methods described herein. Here, engineered cells identified as HEK line #1 exhibited the highest increase in exosome production. Of note, the fold change between HEK line #1 and the WT exosome production is substantial (e.g., 4-fold change). Considering the remining five engineered HEK cell lines, each of the engineered lines exhibited a substantial increase fold change in the production of exosomes due to the mutated mtDNA associated with the intact mitochondria transferred therein. In some examples, mtDNA depleted cells may be configured to increase exosome production without mitochondria transfer, with mitochondria transfer, or a combination thereof.

In some examples, isolated engineered recipient cells may be sequenced using one or more sequencing processes (e.g., next-generation sequencing) to evaluate and provide the genomic data associated with the recipient cell DNA (e.g., mtDNA), RNA, proteins or a combination thereof. For example, genetic material may be extracted from the recipient cells prior to any of the methods described herein. The genetic material may be sequenced and used to compare sequence data from the engineered recipient cells. In some examples, the mitochondria may be extracted and isolated from the isolated engineered recipient cells for sequencing. Sequence data from the from the isolated engineered recipient cells may be extracted and compared against the initial recipient cell sequence data to identify genomic differences associated with the transferred mitochondria.

In some examples, comparative analysis of sequencing data may provide one or more identifiable alterations (e.g., mutations) in the mtDNA attributable to the phenotypic changes observed in the engineered recipient cells. Accordingly, any method described herein may also include the generation of a mtDNA sequence database to include polynucleotide sequences that may be used for synthesizing or targeted engineering of mitochondria in subsequent cell lines.

The methods of transferring mitochondria, as described herein, can be configured to provide for a high-throughput transfer of large cargo, such as intact mitochondria, to evaluate substantially more mutations and alterations in mtDNA and the impacts they have on cellular activity.

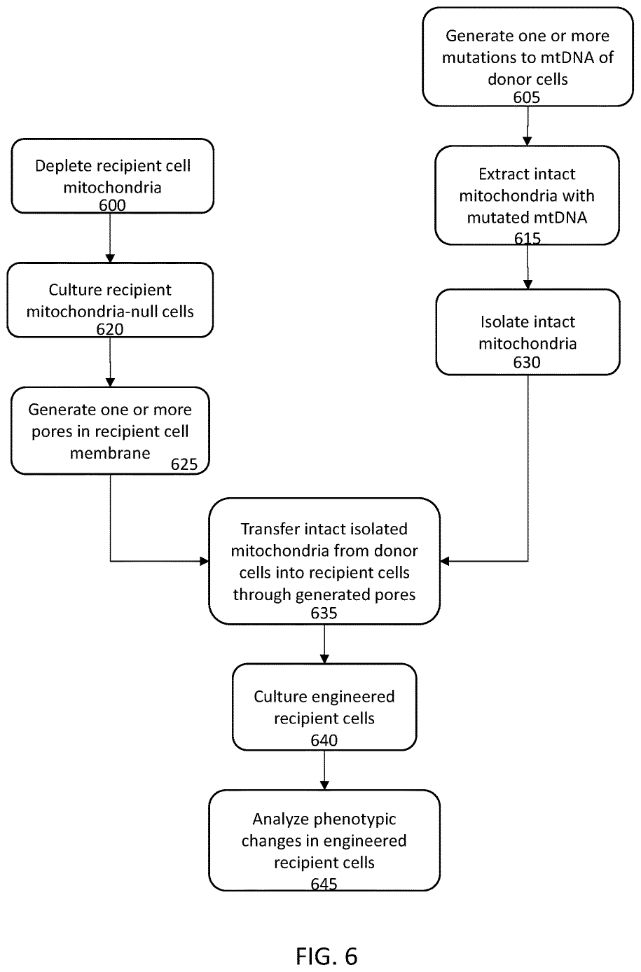

FIG. 6 illustrates an example of methods described herein shown in a flow diagram. The recipient cells and donor cells may be prepared in an independent manner from one another, with the recipient cell workflow shown starting at step 600 and the donor cell workflow starting at step 605.

The donor cells may be subjected to one or more processes to generate mutations in the mtDNA at step 605. As described herein, mutations in the mtDNA may be induced with chemical or other means to generate one or more mutations in the mtDNA. In some examples, the mutations may be pathogenic mutations associated with disease-related changes to the mitochondrial function.

The mitochondria with mutated mtDNA in the donor cells are then extracted at step 615. The mitochondria can be extracted intact such that the entire organelle is extracted from the donor cells.

These extracted mitochondria can then be isolated at step 630 and suspended in an appropriate solution (e.g., PBS).

Turning to the recipient cells, at step 600, the recipient cell mitochondria can be depleted. As described herein, induced mitophagy or other processes may be applied to the recipient cells to sufficiently deplete the endogenous mitochondria in preparation for receiving the engineered exogenous mitochondria from the donor cells.

After the mitochondria has been depleted at step 600, the mitochondria-null recipient cells are cultured at step 620 and subjected to one or more systems or processes configured to generate one or more pores in the cell membrane at step 625. The pores are configured to provide a physical opening in the cell to facilitate the transfer of the intact mitochondria, extracted from the donor cell, into the recipient cell. As described above, a number of known transfer platforms are configured for formation of pores in cellular walls and/or for the preparation of recipient cells for cargo delivery.

At step 635, the mitochondria from the donor cells is transferred into the recipient cells with the transfer platform. The mitochondria remains intact throughout the entire process of mutagenesis, extraction, isolation, and transfer. In some examples, the mitochondria may be substantially intact, or broken into mitochondrial fragments. In some examples, the mitochondria must remain intact throughout the entire method described herein.

After the intact mitochondria has been transferred into the recipient cells, the resulting engineered recipient cells are cultured at step 640, cloned and stabilized. The resulting engineered recipient cells are then analyzed for resulting phenotypic changes at step 645 to evaluate changes in cellular morphology, activity, function, gene expression, etc.

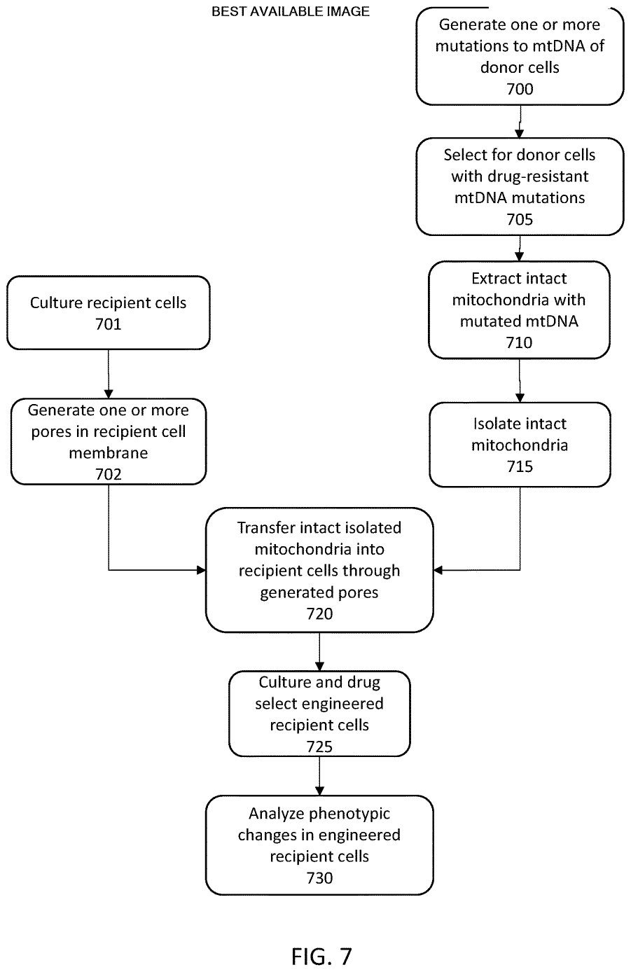

The flow diagram illustrated in FIG. 7 shows an example of a method described herein on the transfer of drug resistant mtDNA from donor cells into recipient cells.

The following illustrate examples of phenotypic modification based on mitochondrial engineering and large-cargo transfer in recipient cell lines. Mutations are generated in donor cells at step 700. For example, mutations in donor cell mtDNA may be generated as a result of a mutated POLG configured to generate one or more random mutations in the mtDNA of the donor cell. In some examples, donor cells with the mutated mtDNA may be selected.

At step 705, for example, mutated donor cells with drug resistant mtDNA mutations may be selected, and the mitochondria from these selected donor cells may be extracted at step 710. In some examples, the mitochondria of the donor cells having drug-resistant mtDNA mutations may be extracted intact, substantially intact, or the mtDNA may be extracted, or a combination thereof.

In some examples of the methods described herein, intact mitochondria extracted from the mutated donor cells may be isolated at step 715.

Recipient cells can be cultured at step 701 and pores may be generated in the recipient cell membranes as illustrated in step 702.

Then, the isolated mitochondria (e.g., isolated intact mitochondria) from the donor cells can be transferred into the recipient cells through the generated pores at step 720, using any of the transfer platforms described herein.

The recipient cells, now comprising the transferred mitochondria from the mutated donor cells are then cultured at step 725 for further analysis.

The resulting engineered recipient cells comprising the mutated mitochondria can then be analyzed for one or more phenotypic changes at step 730. The phenotypic changes in the engineered recipient cells can be based on mutation in the mtDNA of the mitochondria extracted and transferred from the donor cells.

In some examples of any of the methods described herein, recipient cells may be configured to receive mtDNA (e.g., intact mitochondria) from donor cells without depleting the mitochondria of the recipient cells. For example, recipient cells may be configured to receive mutated mtDNA (e.g., via transferring intact mitochondria from mutated donor cells) transferred from donor cell mitochondria comprising one or more drug-resistant mtDNA mutations to generate new mtDNA engineered recipient cell lines. An illustrative example may be certain mtDNA mutations in the donor cell can result in cell resistance to mitochondrial translation inhibitors, chloramphenicol or CAP. Accordingly, selection or isolation of the engineered recipient cells may be done using drug selection (e.g., based on resulting drug resistance).

In a human embryonic kidney (HEK) cell line as recipient cells, the recipient cell line was depleted of mitochondria (e.g., mtDNA) using chemical agents to induce mitophagy. The HEK cells were sufficiently depleted of mitochondria to a mitochondrial-null state. The cells were cultured in an appropriate medium to maintain viability while donor cells were prepared. Donor cells were obtained with POLG deficiency to promote and cause random mutations to the donor cell mtDNA based on the ineffective and inactive polymerase gamma mediated mtDNA repair. Intact mitochondria was extracted from the donor cells and suspended in phosphate buffered saline (PBS) and prepared for transfer into the HEK recipient cells. The HEK recipient cells were cultured on a porous membrane and pressure may be applied to the cell membrane culture sufficient to induce one or more pores in the cellular membranes. Pressure applied to the intact mitochondria and configured to force the intact mitochondria through the pores generated by the chip in the HEK cell plasma membrane.

In some examples, one or more techniques for generating cell lines (e.g., generating pores in cellular membranes) for phenotypic changes caused by mutation sin the mtDNA may include culturing the cells on one or more cell culture platforms, chips, membranes, etc., or a combination thereof. For example, a technique may rely on pressure (e.g., pressurized fluid, gas etc.) and cells may be cultures on a porous membrane. In some examples, heat energy (e.g., using a laser) may be a technique used in developing cell lines and cells may be cultured on a chip (e.g., a silicon chip) configured to receive energy from a pulse laser to increase temperature at one or more areas of the chip. In such an example, the donor cell mitochondria may be positioned adjacent to the chip, and a pulse laser can contact the silicon chip to generate pores in the HEK plasma membrane. In some examples, pressure may be applied to the intact mitochondria that can be configured to force the intact mitochondria through the pores (e.g., pores generated by a heated silicon chip) in the HEK cell plasma membrane. In some examples, pressure may be applied alone or in combination with heat energy. The applied pressure may comprise a pressurized fluid configured to induce pores in the cell membrane and the generated pores can be configured to facilitate mitochondria transfer into the cells, out of the cells, or a combination thereof.

The HEK cells were then cultured and analyzed for phenotypic changes. HEK cells were cloned, mtDNA was stabilized and available for further evaluation (e.g., sequencing). Some examples of resulting phenotypic changes in the HEK cell activity included high impact of 2-3 fold change compared to other engineered HEK recipient cells.

HEK recipient cells exhibited 5 fold change in production of antibodies (e.g., PD1) compared to wild-type HEK cells.

In a cell line derived from epithelial fibroblasts, the native mtDNA was depleted. The fibroblast were treated with a process to induce mitophagy and cause a mitochondrial-null state in the fibroblasts. The fibroblasts were cultured on a chip and pores were generated in the fibroblast plasma membrane. mtDNA (e.g., intact mitochondria) was subjected to pressure (e.g., fluid pressure) sufficient to force the intact mitochondria into the mitochondria-null fibroblasts. The engineered recipient fibroblasts were then cultured, and the mtDNA was stabilized. The stabilized fibroblasts were then analyzed and exhibited an mtDNA genome different than the native fibroblast mtDNA genome. The stabilized fibroblasts were then reprogrammed to a pluripotent state and provided a stabile induced pluripotent stem cell (IPSC) line. The derived IPSC cells were then subjected to differentiation factors providing viable mesenchymal cells having improved phenotypic characteristics compared to the wild-type fibroblasts due to the mitochondrial engineered (e.g., intact mitochondria transfer). In some examples, any of the methods described herein may modulate, regulate, increase, decrease, induce, inhibit, promote, etc. or a combination thereof one or more cellular characteristics (e.g., mitochondrial activity, cytotoxicity function of immune cells, etc.).

In some examples, any of the methods described herein may provide for a fold change in one or more phenotypic characteristics, function, activity, etc. of an engineered recipient cell of less than 1, 1, 2, 3, 4, 5, 10, 15, 20, 30, 40, 50, 60, 70, 80, 100 or more. In some examples, the fold change may relate to the production of an antibody, cytokine, enzyme, hormone, etc.

In some examples, after one or more engineered recipient cells are isolated and the resulting phenotypic change is identified, the isolated engineered recipient cells are cloned. The cloned cells are cultured and grown. The mtDNA is stabilized. Stabilizing the mtDNA may include stabilizing the mtDNA and the nuclear genome of the recipient cell. For example, the mtDNA is based on the donor cell. Accordingly, stabilizing the mtDNA with the nuclear DNA within the recipient cell can improve cellular viability.

In any of the examples described herein, the DNA, mtDNA, RNA, or a combination thereof may comprise one or more pathogenic mutations. In any of the examples described herein, the DNA, mtDNA, RNA, or a combination thereof may comprise one or more non-pathogenic mutations. In any of the examples described herein, the DNA, mtDNA, RNA, or a combination thereof may comprise one or more pathogenic mutations and one or more non-pathogenic mutations. In some examples described herein, the mutations in the DNA, mtDNA, RNA, or a combination thereof may be induced or naturally occurring. In some examples, mutations to DNA, mtDNA, RNA, or a combination thereof may be somatic mutations, germline mutations, environmentally induced or naturally occurring within the cell.

In some examples, a method of engineering a cell, as described herein may comprise identifying changes in phenotype of one or more cells by mutating mtDNA with a mutated nuclear gene (e.g., polymerase gamma). Polymerase gamma may be mutated and configured to cause one or more mutations in the mtDNA. The mutations caused by the polymerase gamma may be analyzed through genomic or phenotypical testing to identify, categorize, isolate, etc. the cells. In some examples, wild-type cells may be selected based on mutation in a gene encoding polymerase gamma. In some examples, one or more mutations to genes encoding polymerase gamma may be induced in a cell and the induced mutations can be configured to provide (e.g., cause) one or more other mutations in the mtDNA. In some examples, mutations in the mtDNA caused by polymerase gamma may be random and result in one or more phenotypic changes. The mutated cells may be analyzed, isolated, evaluated, categorized, separated, or otherwise identified based on the resulting changes in phenotype. In some examples, isolated mutated cells may be subjected to sequencing to identify changes in the mtDNA associated with the phenotypic changes.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of cellular engineering using exogenous intact mitochondria, the method comprising:
removing or depleting endogenous mitochondria from one or more recipient cells;
mutating mtDNA within one or more donor cells;
extracting mitochondria having mutated mtDNA from the one or more donor cells;
transferring the mitochondria having mutated mtDNA into the one or more recipient cells, wherein the mitochondria are transferred intact;
identifying one or more phenotypic changes of the one or more recipient cells, wherein the one or more phenotypic changes is associated with the transferred mitochondria; and
isolating one or more of the recipient cells based on the identified phenotypic changes.

2. The method of claim 1, wherein the step of mutating mtDNA within the one or more donor cells comprises contacting the one or more donor cells with one or more agents configured to modulate one or more mitochondrial polymerases.

3. The method of claim 1, further comprising proliferating the one or more donor cells before extracting mitochondria therefrom.

4. The method of claim 1, further comprising proliferating the one or more recipient cells after transferring the extracted mitochondria.

5. The method of claim 1, wherein the one or more phenotypic changes are caused by the transferred mitochondria.

6. The method of claim 1, wherein the one or more donor cells comprise a mutated polymerase gamma configured to induce one or more mutations in the mtDNA.

7. The method of claim 1, wherein transferring the extracted mitochondria intact into the one or more recipient cells comprises generating one or more pores within a cellular membrane of the one or more recipient cells, pressurizing a flow of the extracted mitochondria into the one or more recipient cells through the one or more pores.

8. The method of claim 1, wherein transferring the extracted mitochondria intact into the one or more recipient cells comprises disrupting a cellular membrane of the one or more recipient cells with a photothermal-based system, wherein the disrupted cellular membrane is configured to permit the transfer of the extracted mitochondria into the one or more recipient cells.

9. The method of claim 7, wherein generating one or more pores within the cellular membrane of the one or more recipient cells comprises growing one or more recipient cells on a porous silicon chip, contacting the silicon chip with a laser configured to increase a temperature of the silicon chip, wherein the temperature is configured to disrupt a cellular membrane of the one or more recipient cells grown thereon.

10. The method of claim 1, further comprising:
cloning the one or more isolated recipient cells; and
sequencing mtDNA from the isolated one or more recipient cells, wherein the sequenced mtDNA comprises one or more different nucleotide sequences than the removed endogenous mitochondria.

11. The method of claim 1, wherein removing the endogenous mitochondria comprises depleting the one or more recipient cells of endogenous mitochondria.

12. The method of claim 1, wherein protein production is increased in the one or more recipient cells based on the transferred mitochondria.

13. The method of claim 1, wherein a growth rate of the one or more recipient cells is increased based on the transferred mitochondria.

14. The method of claim 1, wherein exosome production is increased in the one or more recipient cells based on the transferred mitochondria.

15. The method of claim 1, wherein ATP synthesis is increased in the one or more recipient cells based on the transferred mitochondria.

16. The method of claim 1, wherein one or more metabolic processes is increased in the one or more recipient cells based on the transferred mitochondria.

17. The method of claim 1, wherein protein production is decreased in the one or more recipient cells based on the transferred mitochondria.

18. The method of claim 1, wherein a growth rate of the one or more recipient cells is decreased based on the transferred mitochondria.

19. The method of claim 1, wherein exosome production is decreased in the one or more recipient cells based on the transferred mitochondria.

20. The method of claim 1, wherein ATP synthesis is decreased in the one or more recipient cells based on the transferred mitochondria.

21. The method of claim 1, wherein one or more metabolic processes is decreased in the one or more recipient cells based on the transferred mitochondria.

22-41. (canceled)
